(19) United States
(12) Patent Application Publication
(10) Pub. No.: US 2005/0149202 A1
(43) Pub. Date: Jul. 7, 2005

(54) Liner with exterior coating for use with prosthetic devices

(76) Inventors: Stephen P. Schaffer, Hamburg, NY (US); Barry A. May, Lockport, NY (US); Thomas D. Roman, Niagara Falls, NY (US)

Correspondence Address: DARBY & DARBY P.C., P. O. BOX 5257, NEW YORK, NY, 10150-5257, US

(21) Appl. No.: 10/754,099

(22) Filed: Jan. 7, 2004

(51) Int. Cl. A61F 2/80

(52) U.S. Cl. 623/36; 427/2.31

(57) ABSTRACT

An improved liner (i.e., prosthetic liner) for covering a residual limb of an amputee is provided. According to one exemplary embodiment, the liner includes a fabric liner body that is formed of at least two fabric pieces and a stretch limiting element that is incorporated into an exterior surface of the distal end of the fabric liner body for reinforcement of the liner's distal end and for limiting stresses associated with distal end elongation. For example, the stretch limiting element can be in the form of a coating of a material that has limited flexibility and is integrally adhered to the outer surface of the fabric liner body such that the material seeps into the interior of the fabric liner body and optionally into any seams. Since the coating is integrally adhered to the fabric liner body and has an elasticity that is lower than that of the fabric liner body, the coating serves as a stretch limiting, anti-elongation element which counters forces that tend to result in the fabric liner body being stretched out during normal every day use.

### TECHNICAL FIELD

The present invention relates generally to an article to be worn over an amputee's residual limb, and more particularly, to a liner that includes a surface reinforcement feature for limiting overall stretch of the article and for increasing wear characteristics of the article.

### BACKGROUND

For the past decades, amputees have worn tubular sock-like articles over their residual limbs to provide additional comfort to the amputee when wearing a prosthetic limb. For many years, the tubular sock-like articles were formed of natural materials, such as cotton, wool, and cotton-wool blends; however, as synthetic materials become increasingly popular as a material of choice to form articles of apparel, including socks, the tubular sock-like articles were increasingly fabricated using synthetic materials.

As is known, an amputee is typically fitted with a prosthetic member to be worn over the residual limb. In a below-knee (BK) prosthesis, an amputee's stump tends to pivot within a socket of the prosthesis. During ambulation, the stump will come up in the socket of the prosthesis until the means for attaching the prosthesis to the wearer causes the prosthesis to lift with the stump. The wearer then completes a walking motion or other movement by repeatedly lifting the prosthesis up and then placing it back down in a different location to effectuate movement of the wearer's body.

Most of the available cushioned residuum socks (prosthetic liners) that are currently available have a tubular or conical construction and do not provide a form fit of the amputee's residuum since the residuum stump typically does not contain a completely uniform shape. For example, while the residuum stump generally has a roughly conical shape, the residuum stump will often have recessed areas in certain locations. On a below knee, left side residual limb, the recessed area is often more pronounced on the right side of the tibia bone, while for right side residual limbs, the more pronounced recessed area is on the left side of the bone. In both instances, the side opposite the side with the more pronounced recessed area will also contain a recessed area to a lesser degree and further the greatest recess typically occurs immediately below the patella, on either side. Conventional prosthetic liners do not accommodate the non-uniform nature of the residuum and this can result in the amputee experiencing wearing discomfort due to the non-uniform fit.

When the amputee uses a prosthetic device, the amputee simply attaches a prosthetic limb to his or her residual limb by means of a rigid socket, liner, and a suspension means. The rigid socket can be custom fabricated to match the shape of the intended user's residual limb and can be formed from a variety of different materials, including but not limited to thermoplastic materials, fiber-reinforced thermoset materials, as well as wood and metals. Because the residual limb interfaces with the hard, rigid prosthetic limb, this interface can become an area of discomfort over time since this interface is a load bearing interface between the residual limb and the prosthetic limb. In order to alleviate this discomfort and provide a degree of cushioning to lessen the impact of the load, prosthetic liners (socks) are used as interface members between the hard prosthetic socket and the residual limb in order to increase comfort.

One of the disadvantages of conventional prosthetic liners (socks) is that they are subjected to a variety of different forces during normal use, especially at the distal end portion, such that the wearer is able to feel the cushioned liner stretch at the distal end portion. This stretching can lead to an uncomfortable feel as the wearer is walking or is otherwise in motion. In addition, the rotation of the liner within a prosthetic socket can also contribute to the distal end portion being placed under stress that can contribute to break-down of the liner or otherwise weaken the structural integrity of the article. Not only does this correspond to the user experiencing discomfort but the stretching (elongation) of the article requires the prosthetic device to be replaced more often.

While, a number of different solutions have been proposed to overcome the aforementioned problem, the proposed solutions suffer from a number of associated disadvantages, including some being too overly complex and costly, etc. For example, a number of patents (e.g., U.S. Pat. Nos. 4,923,474 and 5,728,168) propose to either add an outer reinforcement layer that is axially inelestic to the liner construction or construct the liner of two integrally formed sections with the lower distal section being formed of a material that has a greater hardness compared to the material forming the upper section. However, these solutions both involve the specific construction of the liner walls themselves and therefore, requires special tooling, molding techniques, etc. to be used to achieve the stated results. U.S. Pat. No. 6,136,039 discloses a liner in which an elasticity controlling matrix material is provided between an inner layer and an outer layer of the liner; however, again this inclusion complicates the manufacturing process since an additional layer needs to be specifically placed in a precise location.

Thus, there is a need in the art for a prosthetic liner which overcomes the deficiencies of the prior art and is constructed so that it includes a simple feature to further limit stresses of distal end elongation and rotation in the prosthetic socket.

### SUMMARY OF THE INVENTION

An improved liner (i.e., prosthetic liner) for covering a residual limb of an amputee is provided. According to one exemplary embodiment, the liner includes a sock-shaped fabric liner body and a stretch limiting element incorporated into an exterior surface of the distal end of the fabric liner body for reinforcement of the liner's distal end and for limiting stresses associated with distal end elongation. For example, the stretch limiting element can be in the form of a coating of a material that is integrally adhered to the outer surface of the fabric liner body and has less elasticity then the fabric liner body such that it provides surface reinforcement and limits stresses of distal end elongation of the fabric liner body. In one embodiment, the coating is formed 360° around the distal end region of the fabric liner body. The coating can have either a regular or irregular shape, such as a wave pattern, or can be in the form of one or more strips that extend along a length of the fabric liner body. Since the coating is integrally adhered to the fabric liner body and has less elasticity than that of the fabric liner body, the coating serves as a non-elongating element (stretch limiting feature) in that it counters forces that tend to result in the fabric liner body being stretched out during normal every day use. Suitable materials for the coating include but are not limited to: polyurethanes; liquid silicones (polysiloxanes); polyamides; and mixtures thereof.

The liner typically includes a pin receptacle that is attached to the distal end of the liner body on an exterior thereof. In this embodiment, the coating is at least partially applied to the pin receptacle such that the coating extends from the pin receptacle and onto the distal end region of the fabric liner body. More specifically, the coating is formed 360° around a skirt portion of the pin receptacle and extending onto the exterior surface of the fabric liner body.

According to one embodiment, the liner includes a fabric body member formed of at least two fabric pieces. One of the fabric pieces is a distal end piece that is attached to at least one other fabric piece along a circumferential edge of the distal end piece. The distal end piece is free of a transverse seam that extends across the distal end piece. The liner can also include a cushion layer disposed on an interior surface of the fabric member.

Other features and advantages of the present invention will be apparent from the following detailed description when read in conjunction with the accompanying drawings.

### BRIEF DESCRIPTION OF THE DRAWINGS

The foregoing and other features of the present invention will be more readily apparent from the following detailed description and drawings of illustrative embodiments of the invention in which:

FIG. 1 is a perspective view of a sock-shaped liner according to one exemplary embodiment of the invention, placed over a residual limb of an amputee;

FIG. 2 is a perspective view of the liner of FIG. 1 with the residual limb fully inserted into the liner;

FIG. 3 is a partially exploded perspective view of the liner of FIG. 1 with a section of the liner shown in cross-section;

FIG. 4 is an exploded perspective view of a liner body of the liner illustrating exemplary points of attachment between the individual elements;

FIG. 5 is a perspective view of an exemplary fabric taken from circle 5 of FIG. 4 and used to form the individual elements of the liner body of FIG. 4;

FIG. 6 is a cross-sectional view taken from circle 6 of FIG. 3;

FIG. 7 is a perspective view of a liner with the residual limb fully inserted into the liner and the liner including a surface reinforcement feature according to a first embodiment for limiting overall stretch thereof;

FIG. 8 is a perspective view of a liner with the residual limb fully inserted into the liner and the liner including a surface reinforcement feature according to a second embodiment for limiting overall stretch thereof;

FIG. 9 is a perspective view of a liner with the residual limb fully inserted into the liner and the liner including a surface reinforcement feature according to a third embodiment for limiting overall stretch thereof;

FIG. 10 is a perspective view of a liner with the residual limb fully inserted into the liner and the liner including a surface reinforcement feature according to a fourth embodiment for limiting overall stretch thereof;

FIG. 11 is a perspective view of a liner with the residual limb fully inserted into the liner and the liner including a surface reinforcement feature according to a fifth embodiment for limiting overall stretch thereof;

FIG. 12 is a perspective view of a liner with the residual limb fully inserted into the liner and the liner including a surface reinforcement feature according to a sixth embodiment for limiting overall stretch thereof in both the distal and proximal regions;

FIG. 13 is an exploded perspective view of a support element relative to a partially rolled down sock prior to insertion therein;

FIG. 14 is a perspective view of a mask being applied to a supported liner for marking a distal end region to apply a surface reinforcing material;

FIG. 15 is an exploded perspective view of the masked liner with a rotatable lathe in close proximity and for connection to a pin receptacle of the liner;

FIG. 16 is a perspective view of a manual hand held applicator being used to apply the surface reinforcing material to the masked region of the liner;

FIG. 17 is a perspective view of a roller being used to smooth out the surface reinforcing material after it has been applied to the liner;

FIG. 18 is a perspective view of a first automated hand held applicator being used to apply the surface reinforcing material to the masked region of the liner;

FIG. 19 is a close-up of an underside of the applicator illustrating ports for discharging the surface reinforcing material;

FIG. 20 is a perspective view of a second automated applicator with the supported liner being exploded therefrom and prior to insertion therein for coating a selected region of the liner with the surface reinforcing material; and

FIG. 21 is an elevation cross-sectional view of the liner mounted within the second automated applicator illustrating the surface reinforcing material being applied thereto.

### DETAILED DESCRIPTION OF THE PREFERRED EMBODIMENTS

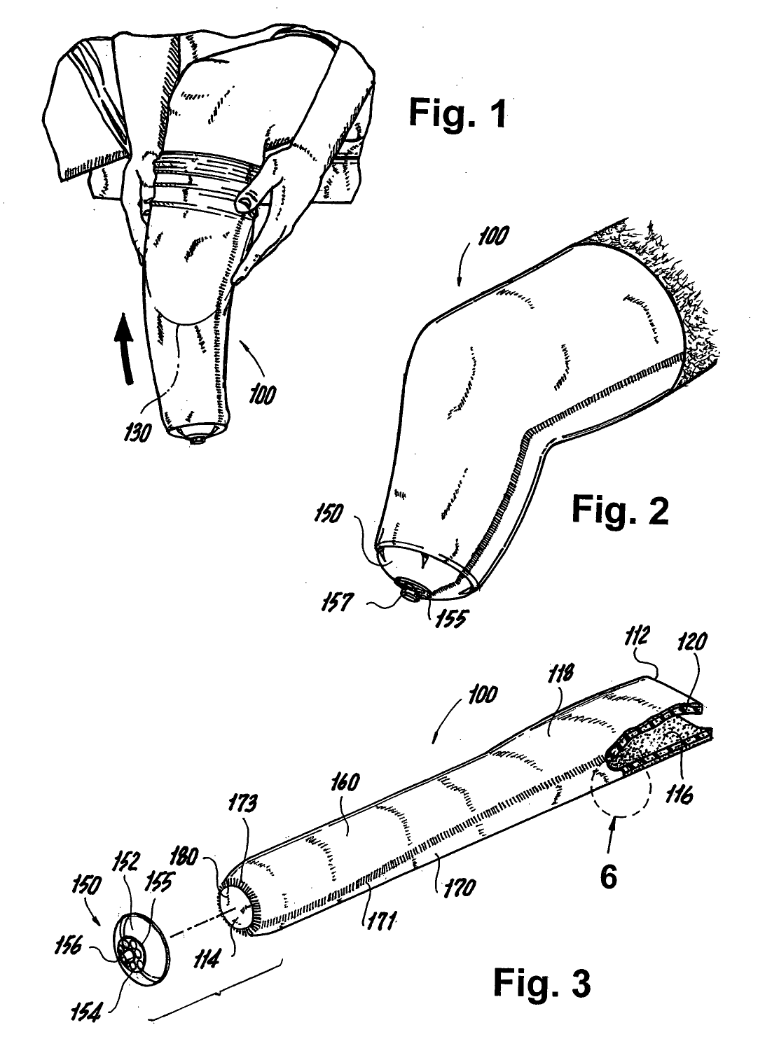

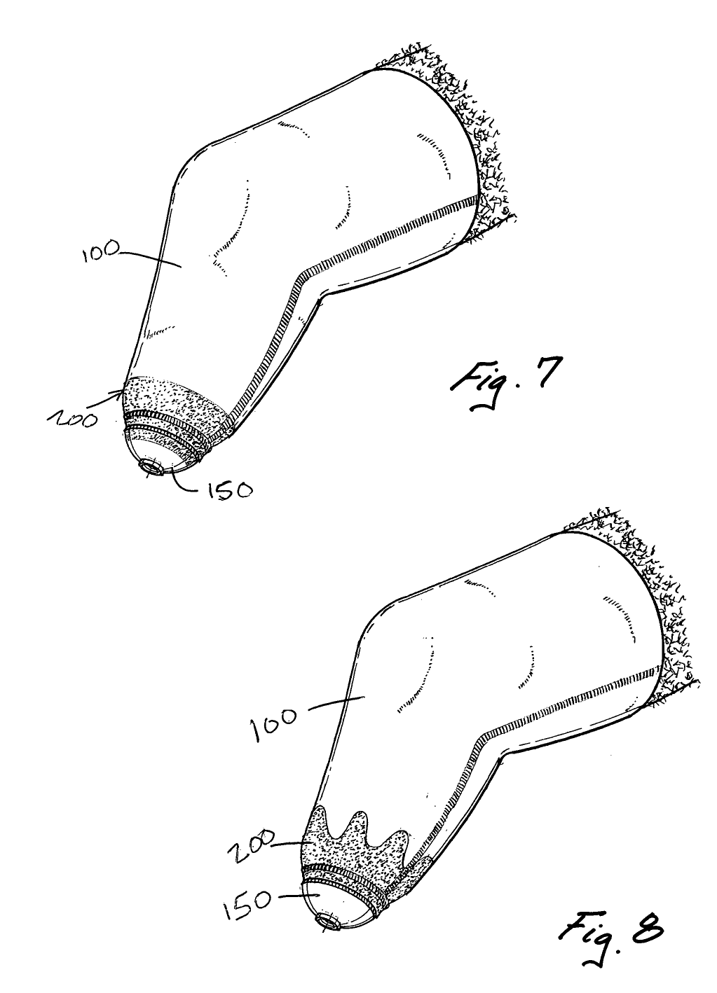

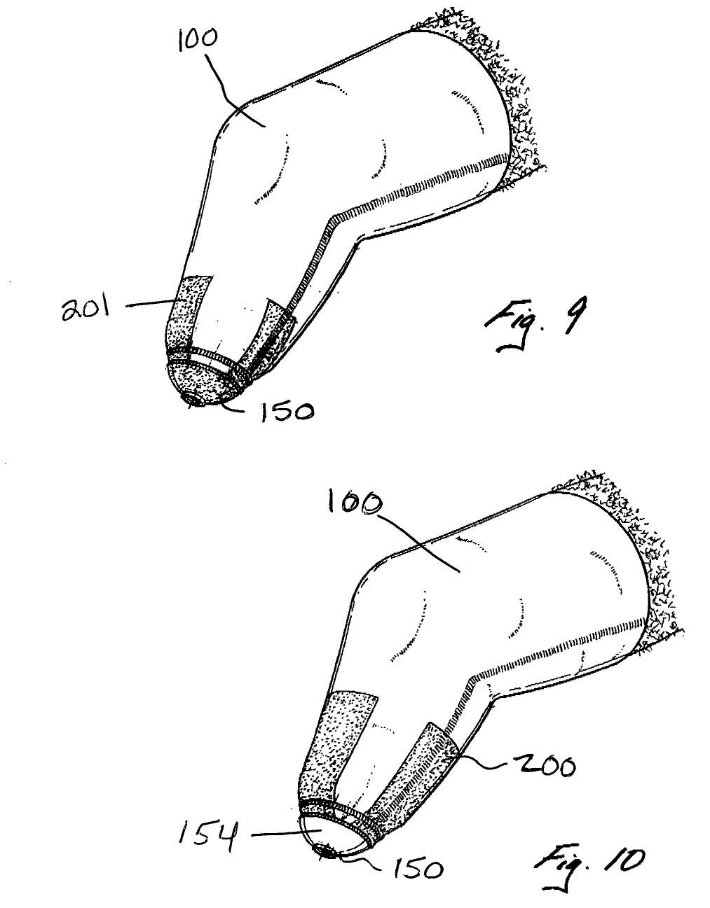

Referring to FIGS. 1 through 6, a liner (i.e., a prosthetic liner) 100 according to one exemplary embodiment is illustrated and is further described in detail in U.S. patent application Ser. No. 10/102,299, filed Mar. 19, 2002, which is hereby incorporated by reference in its entirety. The liner 100 is formed of a liner body 110 having a form fitting generally tubular sock-shape having an open end 112 into which an amputation stump (residual limb) 130 can be introduced, and a closed distal end 114. The liner body 110 includes an interior 116 and an exterior 118; according to one exemplary embodiment, the liner 100 is of a cushioned type, with the interior 116 being impregnated with a cushioning material to form a cushion layer 120 so as to provide a cushion between the amputee's residual limb 130 and a prosthetic device (not shown) which is to be attached to or otherwise coupled to the residual limb 130, as will be described in greater detail below.

When the liner 100 is used to couple a prosthetic device to the residual limb 130, a pin receptacle 150 is preferably provided and is attached to the distal end 114 of the liner 100 on the exterior 118 thereof. In the exemplary embodiment, the pin receptacle 150 has a resilient radial skirt portion 152 surrounding a receptacle body 154. The receptacle body 154 is a rigid member that is preferably formed of metal and includes a threaded bore 156 which threadingly receives a connecting member (e.g., a threaded pin) of the prosthetic device to securely attach the prosthetic device to the liner 100. The radial skirt portion 152 preferably has a diameter that is approximately equal to or less than the diameter of the distal closed end 114 of the liner body 110 so that the radial skirt portion 152 does not extend beyond the peripheral edge of the liner body 110 at the distal end 114 thereof. In other words, there preferably is a smooth radial interface between the radial skirt portion 152 and the liner body 110. However, in some applications, it may be desirable for the radial skirt portion 152 to extend beyond the peripheral edge of the liner body 110.

The radial skirt portion 152 is a flexible member that is formed of a resilient material, such as a polymeric material. The receptacle body 154 can be formed of any number of materials, such as metals, and in one embodiment, the receptacle body 154 is formed of aluminum.

The pin receptacle 150 is disposed at the closed distal end 114 of the liner body 110 using any number of techniques. When the pin receptacle 150 is disposed on the distal end 114, the receptacle body 154 is generally centered about the distal end 114. The receptacle body 154 has an annular base 155 (i.e., radial flange) that surrounds an annular boss 157 that includes the threaded bore 156. Preferably, the radial skirt portion 152 is formed over the receptacle body 154 and the polymeric material forming the radial skirt portion 152 surrounds the outer surface of the annular boss 157. In other words, the only portion of the receptacle body 154 that is exposed is the threaded bore 156 to receive the connecting member of the prosthetic device and establish a connection between the liner 100 and the prosthetic device.

Suitable techniques for attaching the pin receptacle 150 to the closed distal end 114 include but are not limited to using an adhesive material to bond the pin receptacle 150 to the textile material of the closed distal end 114. It will also be appreciated that a molding process can be used to form the radial skirt portion 152 around the receptacle body 154 and at the same time bond the socket 150 to the distal end 114 of the liner 100. For example, the receptacle body 154 can be placed into a mold, along with the distal end 114 of the liner body 110 and then polymeric material can be introduced into a mold cavity, thereby forming the radial skirt portion 152 and attaching the pin receptacle 150 to the liner body 110.

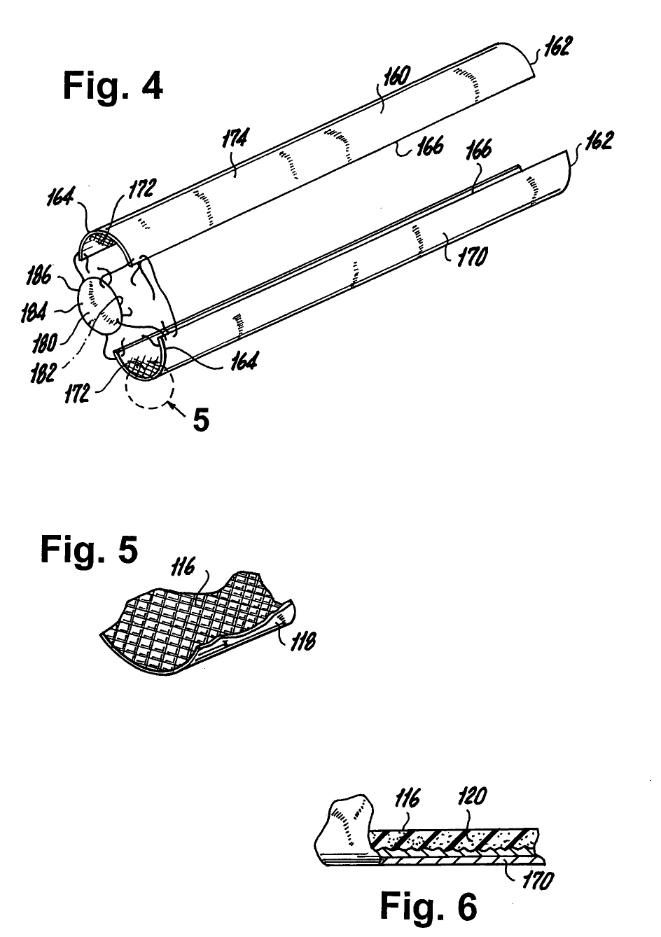

As best shown in the exploded view of FIG. 4, one exemplary liner body 110 is formed of two or more pieces (panels) of textile material that are cut according to an exemplary pattern and then attached to one another along predetermined seams to provide the constructed liner body 110. In one exemplary embodiment, the liner body 110 is formed of three pieces of textile material, namely first and second side panels 160, 170 and a distal panel 180. Preferably, the first and second side panels 160, 170 are identical to one another. Each of the first and second side panels 160, 170 has an upper edge 162 that forms the open end 112 of the liner body 110 when the first and second side panels 160, 170 are attached and an opposing lower edge 164 that forms the closed distal end 114 of the liner body 110.

When each of the first and second side panels 160, 170 is flattened out, each panel has a generally rectangular shape with a slight inward taper toward the lower edge 164. In other words, the upper edge 162 has a width slightly greater than the width of the lower edge 164. Each of the first and second side panels 160, 170 has an interior surface 172 (that forms a part of the interior 116 of the liner body 110) and an opposing exterior surface 174 (that forms a part of the exterior 118 of the liner body 110). As best shown in FIG. 4, the first and second side panels 160, 170 are attached to one another along side edges 166 of each. The side edges 166 extend from the lower edge 164 to the upper edge 162.

The distal panel 180 is a textile piece that is cut to have an annular shape or some other desired shape so long as the distal panel 180 encloses one end of the liner body 110 when it is connected to the side panels 160, 170. The distal panel 180 has an interior surface 182, an exterior surface 184 and a peripheral, circumferential edge 186. The dimensions of the distal panel 180 should be such that when the first and second side panels 160, 170 are attached to one another, the distal panel 180 completely extends across the open lower edge (i.e., defined by the lower edges 162 of the panels 160, 170) so as to enclose the distal end (second end 114) of the liner body 110. Accordingly when the first and second side panels 160, 170 are attached to one another along the side edges 166 to form vertical seams, the liner body 110 has a tubular shape and the distal panel 180 is used to enclose the liner body 110. The distal panel 180 is attached to the lower edges 162 of the first and second side panels 160, 170 along its peripheral, circumferential edge 186.

As best shown in FIG. 5 and according to one embodiment, the interior surfaces 172, 182 of the first and second side panels 160, 170 and the distal piece 180, respectively, have a different texture than the exterior surfaces 174, 184. As will be described in greater detail hereinafter, the textile panels 160, 170, 180 are preferably formed of two different materials that are knit together so that the fibers of one material form the exterior surface of the respective piece and the fibers of the other material form the interior surface of the respective piece. The texture of the interior surfaces 172, 182 is designed to absorb the cushioning material that is applied to the interior surfaces 172, 182 to form the cushion layer 120, while not permitting the cushioning material to bleed through or otherwise migrate to the exterior surfaces 174, 184 thereof. As illustrated in FIG. 5, the exemplary interior surface of the textile material has a waffle-like appearance for absorbing the cushioning material. It will be understood that the liner body 110 can be formed of other fabric materials having different textures that the aforementioned textures. For example, the texture of each side of the liner body 110 can be the same.

Referring now to FIGS. 1 through 6, the two side panels of material 160, 170 used to construct the liner body 110 can be attached to one another using any number of conventional techniques, including stitching the two side panels 160, 170 of textile material along the side edges 166 to form vertical stitched seams 171. When the two side panels 160, 170 are stitched to each other, a wide variety of thread types can be used and a number of different stitch types can be used. In one exemplary embodiment, thread formed of a synthetic material, such as nylon, is used to attach the two side panels 160, 170 to one another using a flat-locked stitch. A flat-locked stitch is preferred because this type of stitch tends to create a smooth seam that is less irritating than seams formed of other stitches. A flat-locked stitch also permits the two side panels 160, 170 to sufficiently stretch to accommodate the stretching of the liner 100 that occurs during the normal wear of the liner 100.

Similarly, the distal panel 180 of textile material can be connected to the distal lower ends 164 of the two side panels 160, 170 along a circumferential stitched seam 173 using any number of stitch types. However, the distal panel 180 of material is preferably connected to the distal lower ends 164 of the first and second side panels 160, 170 of material using a circumferential seam 173 that has a flat-locked stitch.

The first and second side panels of material 160, 170 and the distal panel 180 can be formed of any number of different textile materials having a predetermined thickness (ply). Preferred textile materials are textile fabrics that have an elasticity that permits the prosthetic liner (liner 100) to stretch a predetermined amount during normal application of the liner 100 to the residual limb 130 and during the normal motions of the cushioned liner 100 as the wearer takes steps or otherwise moves the prosthetic limb (i.e., the prosthetic device). For example, the two side panels 160, 170 and the distal panel 180 can be formed of fabrics selected from the group consisting of: stretchable non-woven fabrics (e.g., the Xymide line of fabrics including Wearforce® fabrics from DuPont, Wilmington, Del.); Lycra® based materials which include segmented elastomeric polyurethane fibers (i.e., spandex type fabrics); supplex nylon, neoprene fabrics (polychloroprene fabrics); nylon, spunbonded olefin; looped nylon; spunlaced fabrics; polyester; polypropylene; and aramid fiber fabrics. It will be appreciated that the above list of suitable fabric materials is not exhaustive and is merely exemplary in nature and not limiting of the types of fabric materials that be used to form the liner body 110. Further, it will be appreciated that the fabrics used to form the present liner body 110 are preferably elastic fabrics that can be provided in a woven, knitted, or non-woven form.

One preferred fabric material that is used to form the two side panels 160, 170 and the distal panel 180 is a fabric formed of polyester and polypropylene knit fibers. As shown in FIG. 5, the fabric is constructed (knit) in such a way that the polyester fibers form one surface of the fabric and the polypropylene fibers form the opposite surface of the fabric. In the present prosthetic liner, the polyester surface is intended to form a part of the exterior 118 of the liner body 110, while the polypropylene surface is intended to form a part of the interior 116 of the liner body 110. The polypropylene surface has a distinct texture in that it has a waffle-like texture. When the fibers are knitted in this manner (waffle-like), a number of interstices are formed across the polypropylene surface. As will be described in greater detail hereinafter, the interstitial characteristic of the side of the fabric that forms the interior of liner body 110 advantageously permits gel that is applied to the interior of the liner body 110 to be readily absorbed within the interstices. This type of fabric is commercially available from Milliken & Company of Spartanburg, S.C. under the style/pattern No. 952561-804. Advantageously, a fabric constructed in the aforementioned manner allows the cushioning material to enter into the denier of the fabric but is resistive to the cushioning material migrating through the fabric from the interior surface 116 across the exterior surface 118. In the final product, the cushioning material should be confined to the interior surfaces 116, while the exterior surfaces 118 are free from the cushioning material.

The material used to form the liner body 110 is preferably elastic (stretchable) in one or more, preferably two, directions and is capable of adjusting to variations in form and size of the residual limb 130. Depending upon the precise application, the thickness of the textile material (e.g., fabric) can be altered and while in one embodiment, the material/material thickness of each of the first and second side panels 160, 170 is the same as the material/material thickness of the distal panel 180, it will be appreciated that the material and/or material thickness of any one of the first and second side panels 160, 170 and the distal panel 180 can be different from the other pieces. In one exemplary embodiment, the thickness of the fabric material used to construct the liner body 110 is from about 0.010 inch to about 0.200 inch. In the embodiment where the fabric material is a knit of polyester and polypropylene fibers, the thickness of the fabric material is about 0.04 inch; however, the thickness of the polyester/polypropylene knit can vary depending upon the particular application.

It will be appreciated that the distal end 114 of the liner 100 does not include a seam that is positioned in a location where the residual limb 130 will come into contact therewith. In many cases, the residual limb 130 tapers inwardly toward its distal stump end due to the natural shape of a leg and as a result of typical surgical techniques that are employed during an amputation procedure. The residual limb 130 thus rests against the cushion layer 120 in an area that is within the circumferential seam 173 or at least preferably contacts the circumferential seam 173 at the most peripheral portions of the residual limb 130. At the very least, the wearer of the present liner 100 does not experience a distal seam running across underneath the residual limb 130 and preferably, the liner 100 is constructed so that the contact between the residual limb 130 and the circumferential seam 173 is negligible or nonexistent. As previously-mentioned, this distal end of the residual limb 130 is an extremely sensitive area and therefore, the elimination of any stitching across this sensitive area, provides a cushioned liner that is substantially more comfortable than traditional prosthetic liners.

The cushioning material is applied to the interior surfaces 172, 182 of the first and second side panels 160, 170 and the distal panel 180, respectively, to form the cushion layer 120. The process of applying the cushioning material and controlling the thickness of the cushioning material, so as to permit contouring of the cushioning material, along the interior surface 116 of the liner body 110, is described in great detail in the aforementioned '299 patent application. Preferably, the cushioning material is applied to interior surfaces 172 of the first and second side fabric panels 160, 170 to coat these panels from the lower edge 164 to the upper edge 162 and is also applied to the interior surface 182 of the distal panel 180.

As previously-mentioned, the liner 100 preferably includes the layer 120 of cushioning material and has a form fitting shape with an open end 112 into which the amputation stump 130 may be introduced, a closed end opposite the open end, an interior and an exterior. The interior of the liner 100 is defined by the interior surfaces 172, 182 of the panels 160, 170, 180 that are attached to one another and the interior surfaces 172, 182 are impregnated with a cushioning material to provide a cushion (e.g., cushion layer 120) between the amputee's residuum 130 and any prosthetic device to be worn, attached to, etc., the residuum 130.

The cushioning material is preferably a polymeric material and in one exemplary embodiment, the cushioning material is formed of a gel, a thermoplastic elastomer, or a combination thereof. For example, suitable thermoplastic elastomers include but are not limited to thermoplastic rubbers, silicone (polysiloxane)—containing elastomers, thermoformable materials, etc., that provide a comfortable interface between the residuum 130 and a prosthetic device.

In one exemplary embodiment, the cushioning material is a polymeric gel that is composed of a block copolymer and mineral oil. The gel that can be used to form the cushioning material can either be a nonfoamed gel or a foamed gel (which is produced using a foaming agent). The mineral oil is present in an amount that is effective to produce a cushioning material having desired properties and is preferably present in from 0-85% by weight based on total weight, depending upon the precise application. However, it will be appreciated that in some applications the mineral oil can be present in an amount greater than 90% by weight. One exemplary range for the mineral oil is from about 80% to about 90% by weight.

The polymeric material used to form the liner 100 is characterized by a certain durometer range. According to one exemplary embodiment, durometers for the cushioning material range from 1-20 on the Shore “A” scale. The lower the Shore A number, the softer the material, typically due to a higher level of plasticizer. Preferably the polymeric gel has a durometer (Shore A) that matches or approximates human skin and it has been found that the above durometer range of 1-20 generally provides the gel material with suitable characteristics. In one embodiment, the mineral oil is present on an equal weight basis, or in a weight ratio of 1/4, with regard to the amount of polymeric material present. The mineral oil is preferably purified mineral oil and is preferably USP grade. Once again, the aforementioned ratios and ranges are merely exemplary and thus do not serve to limit the present invention.

In one exemplary embodiment, the cushioning material is formed of a Kraton®-type rubber material (Shell Chemical Co.). For example, the polymeric material can be formed of the following Kraton® rubbers: styrene-ethylene/butylene-styrene block copolymers or styrene-ethylene/propylene block copolymers and are available in triblock and diblock form.

The polymeric cushioning material can also be a blend of Kraton® rubbers and oils, such as mineral oils, (including typical stabilizers) which provide an average durometer of from 1-20. These blends typically are formed of a rubber having a lower durometer (1-10 of the Shore “A” scale) and a rubber having a higher durometer (e.g., 11-20). The blends are preferably capable of being stretched 100% or more before tearing and are capable of providing a form fit to the residual limb due to their inherent elasticity. Further, low durometer Kraton® rubbers and other materials tend to provide the cushioning material disposed of the interior 116 of the liner 100 with a sticky feeling which enhances the ability of the liner 100 to be form fitted against the residual limb 130 due to the intimate contact between the cushioning material and the skin.

In one exemplary embodiment, the polymeric material is a styrene isoprene/butadiene block copolymer or styreneethylene/butadiene-styrene block copolymer. Suitable polymeric materials, having the aforementioned desired properties, are commercially available from a number of sources. For example, polymeric materials commercially distributed under the trade names C-Flex 1970-W5 (R70-339-000), C-Flex 1960-W5 by Consolidated Polymer Technologies of Largo, Fla. and under the trade name Kraton G1654 by Shell Chemical Co. are suitable for use in producing the cushioning material.

The ratio of polymer to mineral oil will vary depending upon the precise application and upon the desired characteristics of the liner 100. Generally, the ratio of polymer to mineral oil can be from about 1:1 to about 4:1. In addition to using styreneisoprene/butadiene or styrene-ethylene/butadiene-styrene block copolymers (mixed with mineral oil), other suitable polymeric materials include styrene-butadiene-styrene and any thermoplastic elastomer or thermoformable material that is capable of being blended with mineral oil and can perform the prescribed function of providing a cushioning material suitable for use in the intended applications. Mixtures of all of the aforementioned polymers can also be used to form the polymeric cushioning material. Again, the aforementioned ratios are merely exemplary and not limiting.

In one preferred exemplary embodiment, the cushioning material is a polymeric material that has gel-like characteristics and is formulated as a blend of a polystyrene-poly(ethylene-ethylene/propylene)-polystyrene block copolymer (SEEPS) and oil, such as one or more mineral oils. A suitable gel-like cushioning material formed of a SEEPS copolymer/mineral oil blend is commercially available under the trade name PolyGel 51299 from PolyGel LLC of Whippany, N.J.

The cushioning material is also selected so that the liner 100 can be placed on the residual limb 130 in such away that the polymeric material does not drag against the skin. For example, it is desirable for the liner 100 to be capable of being rolled before the liner 100 is placed on the residual limb 130 and/or prosthetic device. Advantageously, the cushioning material is also designed to provide beneficial moisture to the residual limb 130 during the wearing of the liner 100. Moreover, the cushioning material may include antioxidants, such as vitamins A, B, and C or any other antioxidants commonly used in polymers. In addition, skin conditioning agents can be added to the polymeric material of the liner 100 to soothe the skin of the residuum during wear. Such skin conditioners include mineral oil, baby oil, etc., which can be added to the polymeric material prior to its application to the liner body. Also, astringents, biocides, medicaments, etc., can be added or applied to the cushioning material to avoid infection or heal sores, etc.

It will be appreciated that the liner 100 of the present invention does not have to include the cushioning material but rather can be constructed without the cushioning material as disclosed herein.

According to the present invention, the liner 100 includes a surface reinforcement element 200 (see e.g., FIGS. 7-12) that serves to limit the elasticity of the cushioned liner at its distal end portion and thereby, increase wear characteristics of the liner 100. More specifically, it is preferred to limit the stretch of the liner 100 at the distal end portion since during normal use, the wearer is able to feel the cushioned liner stretch at the distal end portion. This stretching can lead to an uncomfortable feel as the wearer is walking or otherwise in motion. It is therefore desirable to limit the degree of elasticity at the distal end portion by adding a simple yet effective “anti-stretch” or “non-elongation” element 200 to the liner 100.

It will be understood that the surface reinforcement element 200 can be added to any number of different types of liners, including cushion or pin suspension liners, and is not limited to the illustrated cushion liner 100. Moreover, the surface reinforcement element 200 can be applied to sleeves (a body that is open at both ends as opposed to a liner which is open at one end) in the form of vertical panels that are formed vertically along the sides of sleeve to limit stretch in the vertical direction. Thus, the liner 100 is merely exemplary of one type of liner that the element 200 can be incorporated into to provide the desired stretch limiting fabric reinforcement.

According to one exemplary embodiment, the surface reinforcement element 200 is a coating of a suitable material that is applied to the distal end of the liner 100 in pre-selected locations that results in the liner 100 having increased rigidity in these areas, thereby providing anti-stretch performance.

Any number of different materials are suitable for use as the coating 200 so long as the material is capable of being sufficiently adhered to the fabric of the liner 100 and the material acts to strengthen the fabric in the areas where it is applied and contains the following properties which are advantageous for the intended use.

Suitable materials include polymeric materials that can be adhered to the fabric of the liner 100 by effectively penetrating the fabric; are sufficiently flexible to permit the liner 100 to be inverted; and result in a coating having a durometer hardness of between about 10 and about 70 (shore A). In addition, the coating material should also have the following physical properties: (1) the thickness of the coating 200 should be on the order of about 0.010 inch to about 0.090 inch; (2) the coating material should have an ability to change shape by masking; (3) the coating material should have the ability to be easily turned inside out for donning purposes; and (4) the coating material must strengthen the fabric liner 100. The viscosity of the coating material varies depending upon the type of material being used; however, suitable materials can have viscosities anywhere from less than about 100 cps to about 7,000 cps. However, it will be understood that these are merely exemplary values.

Exemplary materials include but are not limited to polyurethanes; liquid silicones (polymethyl siloxanes); polyamide adhesives; liquid rubber or; polyurethane latices; hot melt adhesives; and any other liquid elastomers having the aforementioned characteristics and suitable for use with the prosthetic liners for the intended purpose. Some exemplary coating materials include but are not limited to: (1) G.E. Silicones (LIM6030A) heat cure silicone rubber; (2) Henkel (Macromelt OM 633) polyamide adhesive; (3) Henkel (Macromelt OM 641) polyamide adhesive; (4) Henkel (Macromelt OM 652) polyamide adhesive; (5) Henkel (Macromelt OM 6208s) polyamide adhesive; (6) Henkel (Macromelt OM 638) polyamide adhesive; (7) Mace (47-268-clear top coat) solvent based polyurethane; (8) Mace (70-181-1 polyurethane solution) solvent based polyurethane; (9) Mace (83-282-1) toluene/isopropanol based reacted polyester polyurethane; (10) Mace (TS 5710 10 Nitrile) solvent based polyurethane; (11) Polytek (74-40) 40 durometer RTV liquid rubber; (12) Polytek (liquid latex 60) natural latex alkaline dispersion; (13) Polytek (Poly 75-60) 60 durometer RTV liquid rubber; (14) Polytek (Polygel 40) 40 durometer RTV brush on rubber; (15) Polytek (Polygel 74-30) 30 durometer RTV liquid rubber; (16) Polytek (Polygel 74-45) 45 durometer RTV liquid rubber; (17) Quantum Silicones (28-77-1) 20 durometer room temperature addition cure silicone rubber; (18) Quantum Silicones (QM-230) 30 durometer room temperature addition cure silicone rubber; (19) Rust-Oleum (Grip Guard) textured rubber coating; (20) Soluol (Solucote 1013M) solvent-free aqueous anionic polyurethane dispersion; (21) Soluol (Solucote 1017) solvent-free aqueous anionic polyurethane latex; and (22) Zeon (Nipol LX552) synthetic NBR Latex.

It will further be appreciated that accelerators can be added to appropriate coating materials to decrease the time period necessary for curing or setting of the materials. For example, some polyurethanes can cure at room temperature over an extended period of time; however, the addition of an accelerator following by curing in a heater, such as an oven, can reduce the curing time to about 1/2 hour or even a matter of minutes, depending upon the material. Once again, the precise curing times and the curing conditions, e.g., temperature, vary depending upon the material that is being used and it may be as short as less than 1 minute at an elevated temperature to as long as overnight at room temperature.

Advantageously, there are a number of application variables that permit the coating 200 to be formed in a number of different settings by using a number of different techniques. For example, the following are just some of the application variables: (1) the coating material can be applied under ambient conditions; (2) the coating material can be applied in a vacuum; and (3) the use of various shapes and designs for the coating 200 permit a desired result to be achieved.

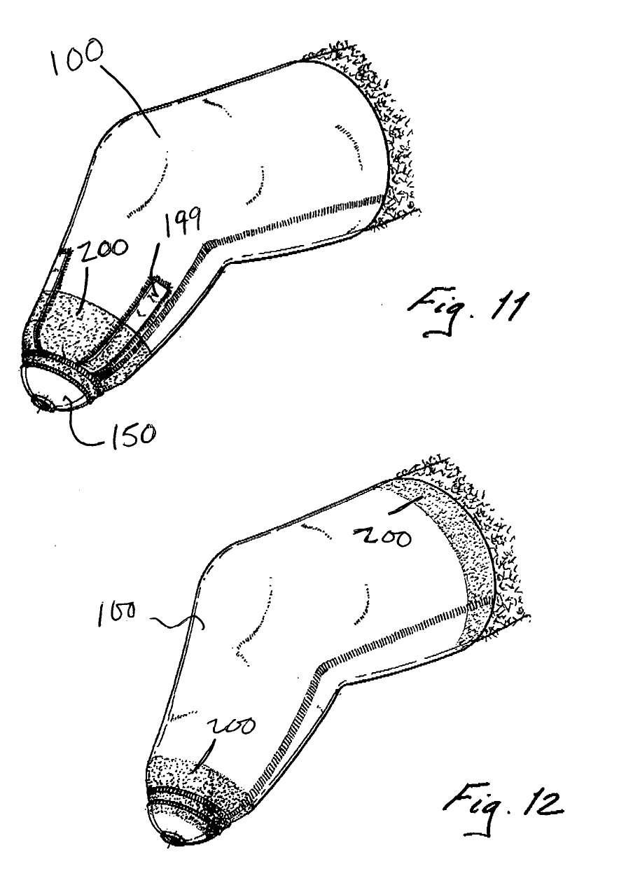

With respect to the above point (3), the design of the coating 200 is highly variable since masks can be constructed to have any number of different shapes and designs and this directly results in variability of the coating 200. For example, FIG. 8 illustrates the coating 200 having a different alternative design. In this embodiment, the coating 200 is formed to have a wave pattern. More specifically, the coating material is disposed up to or over the pin receptacle 150 and the portion of the coating material that is adhered to the liner 100 is the portion that has the wave-like pattern. In other words, the inner edge of the coating 200 is not linear in nature but rather, the inner edge has a series of waves (peaks and valleys) formed as a part thereof any irregular, non-uniform pattern. While the embodiment in FIG. 8 shows the inner edge of the coating 200 as extending to but not over the circumferential edge of the pin receptacle 150, it will be appreciated that the inner edge of the coating can lie over the pin receptacle 150. In other words, a portion of the coating 200 is formed on the pin receptacle 150 as shown in FIG. 7.

In yet another embodiment illustrated in FIG. 9, the coating 200 can be in the form of discrete spokes that extend up along a length of the liner 100 from the pin receptacle 150. For example, the coating 200 can extend circumferentially around the pin receptacle 150 but instead of extending circumferentially around the liner 100, the mask can be constructed so that a majority of the liner is blocked off with only a predetermined number of radial spokes 201 being exposed for receiving the coating material. While the spokes 201 can be formed parallel to one another and perpendicular to the inner circumferential edge of the pin receptacle 150, the spokes can be equally formed so that they extend away from the pin receptacle 150 at an angle.

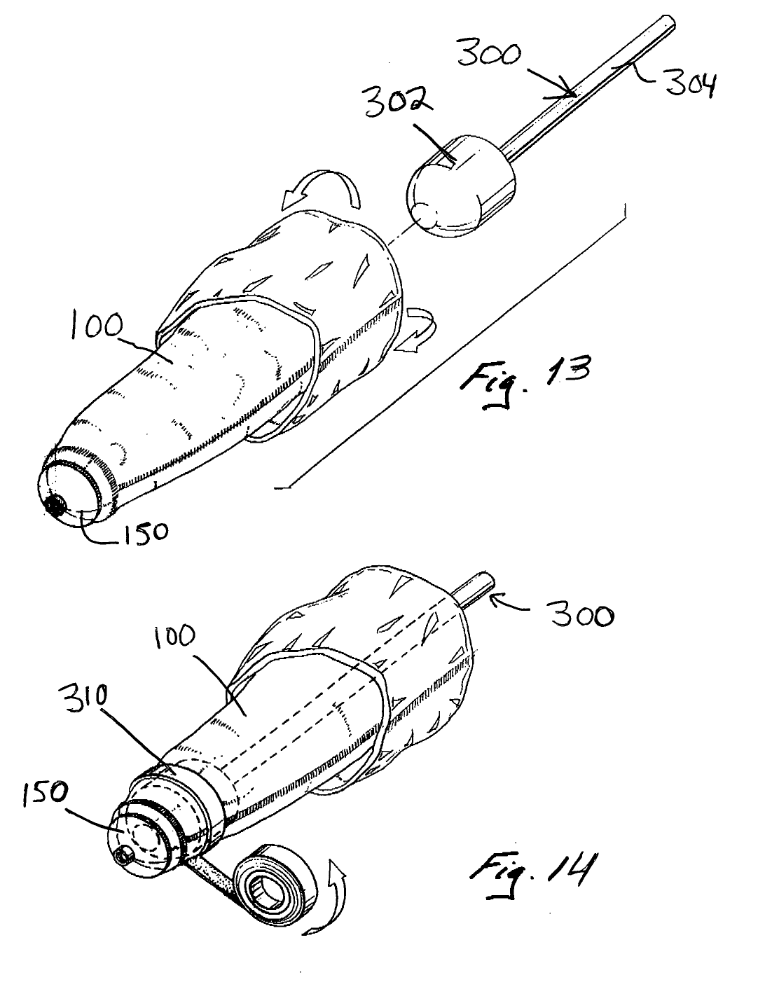

In yet another embodiment shown in FIG. 10, the coating 200 can be in the form of one or more discrete strips that are formed along a length of the liner and extend upwardly from the inner circumferential edge of the pin receptacle 150 and in contrast to the above embodiment, the coating 200 does not necessarily have to be disposed completely circumferentially around the pin receptacle 150 nor does it have to lie on the pin receptacle 150 itself. Instead, one end of one of the discrete strips formed of the coating material can be abutting or proximate to the body 154 of the pin receptacle 150 and the strip extends over the pin receptacle, over its inner circumferential edge and then extends along a length of the fabric liner 100. Thus, strips that are generally rectangular in shape can be formed in the foregoing manner. It will be understood that the coating strips 200 can be formed so that one end thereof does lie on the body 154 of the pin receptacle 150. Preferably, at least some of the coating strips 200 cover the vertical seams that are formed between the joined sections of the liner 100. The lengths and widths of the coating strips 200 are variable depending upon the particular application.

FIG. 11 illustrates yet another embodiment of the present invention. In this embodiment, the stretch limiting element is actually a combination of two elements, namely a plurality of fabric straps 199 that are stitched (sewn) or otherwise attached to the liner 100 and extend up the liner 100 from a location near the pin receptacle 150. The surface characteristic of the liner 100 is altered by incorporating the straps 199 into the liner 100 since the straps are formed of a material that has less elasticity than the liner 100. Thus, the fabric straps 199 serve as stretch limiting elements by limiting or reducing the elongation of the liner 100 in the up and down direction of the liner 100. The second stretch limiting element is the coating 200 similar or identical to that which is described hereinbefore. For example and as illustrated, the coating 200 is a 360° coating that is formed on the liner 100 including over the fabric straps 199. In one embodiment, an inner edge of the coating 200 abuts the circumferential edge of the pin receptacle 150 with the opposite edge lying somewhere between the two ends of the straps 199. However, it will be appreciated that the inner edge of the coating 200 can extend onto and lie over the pin receptacle 150 as previously described herein.

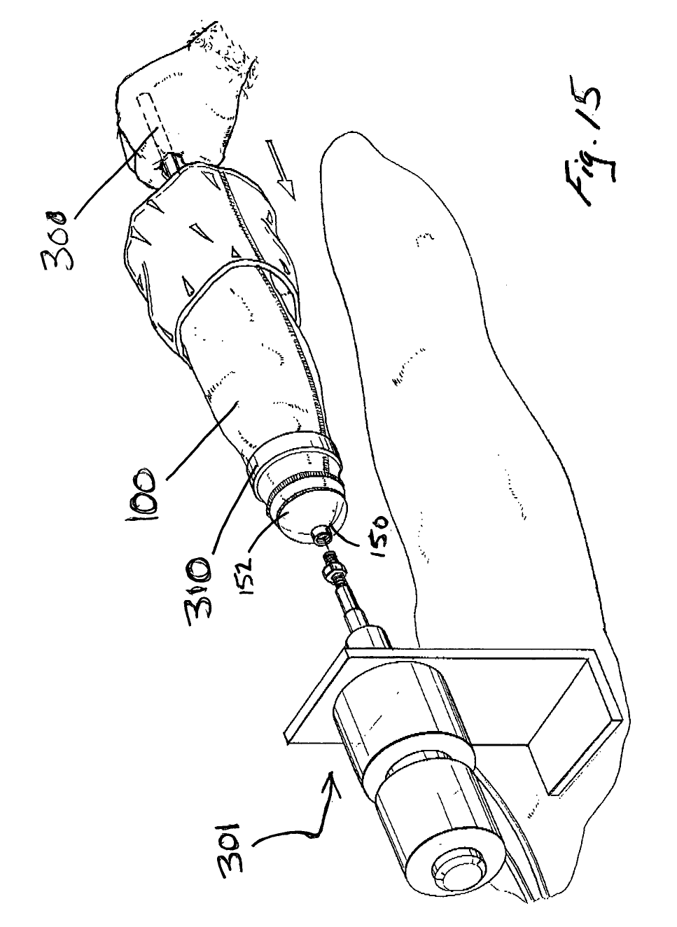

FIG. 12 illustrates yet another embodiment where the surface reinforcement coating 200 is provided at or near a proximal end of the liner 100 as well as being optionally provided at the distal region as previously described. In the exemplary embodiment illustrated, the coating 200 at the proximal region of the liner 100 is in the form of a coating that extends laterally around the liner 100. In other words and as illustrated, the proximal coating 200 is preferably in the form of a lateral band of coating material (similar to the band at the opposite distal region). The lateral band of material can either extend 360° around the liner or the band can extend less than 360° around the liner 100. For example, the lateral band can extend generally 180° around the liner 100 and can be positioned so that it acts either as an anterior coating band or a posterior coating band depending on whether it is placed on the back or front of the liner 100. By placing a lateral band of coating material at the proximal end of the liner 100, the amount of circumferential stretch can be limited. This keeps the liner from stretching out and becoming loose in the proximal area or region of the liner 100. It will be appreciated that the proximal band can take any number of different shapes and sizes and is not limited to the illustrated embodiment. For example, the proximal band can have a wavy pattern as shown in FIG. 8 or any other irregular or regular shape. Also, the coating 200 can take the form of a number of separate lateral bands that are spaced apart from one another. In this embodiment, the lateral band can appear as spaced rectangular shaped blocks of coating.

One exemplary application of the coating 200 is now described in greater detail with reference to FIGS. 13-17. As shown in FIG. 13, the liner 100 is prepared for the application and formation of the coating 200 by first supporting the liner 100 on a support member 300. For example, the liner 100 can be at least partially turned inside out and placed on a support mandrel or the like 300. The support mandrel 300 has a head 302 that has a shape complementary to the interior of the liner 100 and is dimensioned so that it can be received into the interior of the liner 100. The head 302 is attached to an elongated body portion 304 that can be of a pre-selected length. The head 302 thus provides support and serves to spread out the distal end of the liner 100 to facilitate the spreading of the coating 200 about the outer surface of the liner 100. The support mandrel 300 is preferably operatively coupled to a member that causes the selective rotation of the support mandrel 300 at a predetermined speed. For example, the body portion 304 of the support mandrel 300 can be connected to a motorized lathe or the like to rotate the support mandrel 300. The speed at which the support mandrel 300 (and thus the liner 100 that is fitted thereon) is rotated is variable depending upon the particular application and the type of applicator that is being used to apply the coating material on the liner 100. In one embodiment, the support mandrel 300 is rotated at a speed between about 20 and about 60 RPM, e.g., about 30-40 RPM.

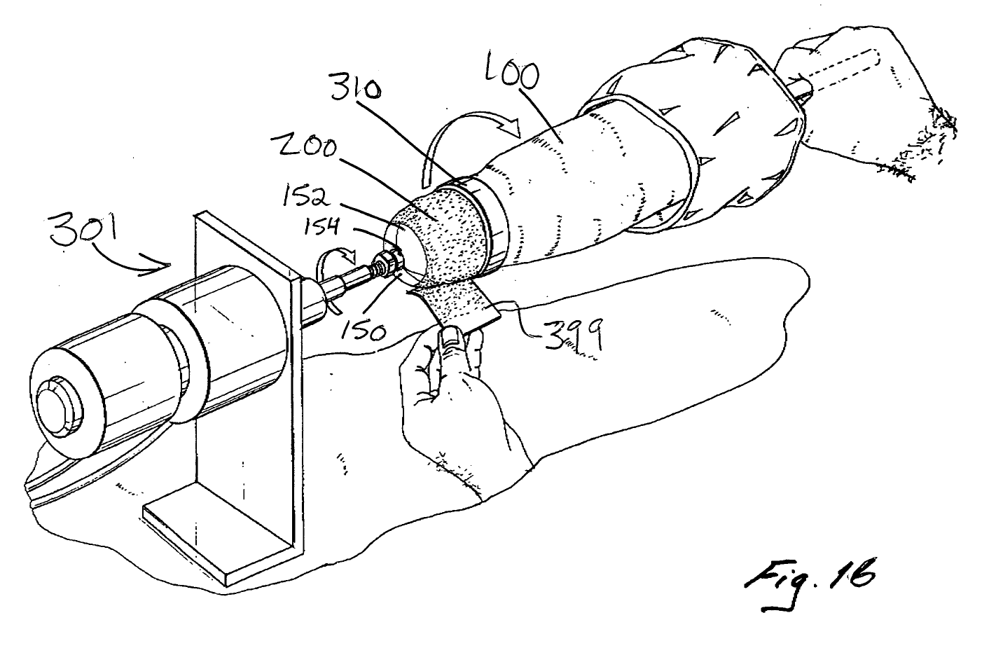

Next, a mask 310 is applied to an outer surface of the liner 100 to cover a portion of the liner 100 and to leave a pre-selected area of the liner 100 uncovered for application of the coating 200 as shown in FIG. 14. In the illustrated embodiment, the mask 310 extends completely around the circumference of the liner 100 and is in the form of a strip of material that is placed proximate the distal end of the liner 100 and it partitions the liner 100 into different areas with the distal end region of the liner 100 being uncovered. More specifically, the mask 310 is placed so that a length of the distal end region of the liner 100 (e.g., between about 1/2 inch to about 4 inches) and the radial skirt portion 152 of the pin receptacle 150 are uncovered and exposed for receiving the coating material. The mask 310 can be formed from any number of different materials so long as it functions to cover and prevent the coating material from being applied to areas of the liner 100 where it is not desired for coating 200 to be formed. In one exemplary embodiment, the mask 310 is a strip of material that completely circumscribes an outer surface of the liner 100 as shown in FIG. 14. In this embodiment, the mask 310 can be thought of as a 360° degree mask and it will therefore be appreciated that the resulting coating 200 that is formed likewise extends 360° around the liner 100. The size of the mask 310 itself is not critical so long as the mask demarcates the area on which the coating 200 is to be formed.

FIG. 15 is a perspective view of the masked liner 100 supported by mandrel 300 being positioned proximate to a device 301 to which the mandrel 300 is adapted to be operatively coupled to such that the mandrel 300 can controllably and selectively be driven. In one embodiment, the device 301 is a rotatable lathe that is configured to be operatively attached to the pin receptacle 150. The rotating device (lathe) 301 is actuated so that the liner is rotated at a speed between about 30-40 RPM.

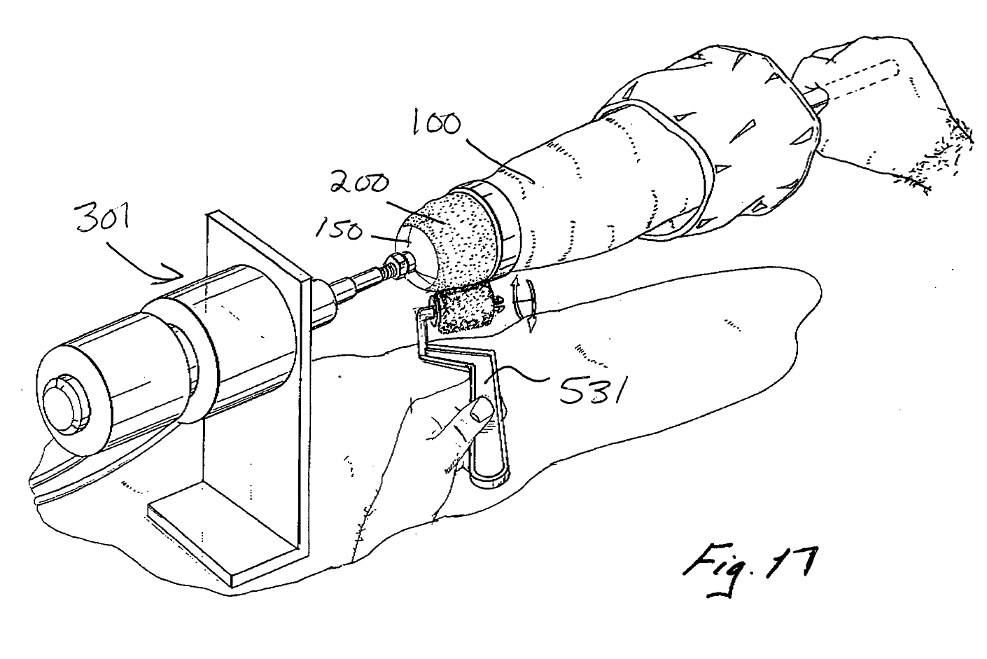

After the mask 310 is placed in its proper position on the liner 100 and the mandrel 300 is attached to the device 301, the coating material 200 is applied to the unmasked region that encompasses a length of the distal end region of the liner 100 and the pin receptacle 150 as shown in FIG. 16. The coating material 200 can be applied using any number of different applicators and applicator techniques so long as the coating material is applied against the liner 100 with sufficient force such that at least a portion of the coating material enters the interstices of the fabric resulting in the coating material being effectively anchored to the liner 100. In other words, the coating material at least partially seeps into the interior of the fabric and the seams of the liner 100 since this is the means by which the coating 200 adheres and strengthens the liner 100. The material that forms the coating 200 may also be of the type that is able to adhere to the radial skirt portion 152 of the pin receptacle 150 when it is so desired; however, this is not a requirement. In the illustrated embodiment, the coating material is disposed all over the radial skirt portion 152 up to and around the base of the receptacle body 154.

One rudimentary method of applying the coating material is illustrated in FIG. 16 and includes preparing a batch of the coating material and storing it in a suitable container and then placing (e.g., dipping) an applicator 399 into the container to collect an amount of coating material on an applicator surface as the user holds the applicator 399. The user then simply presses the coating material that is on the inner applicator surface against and into the fabric liner 100. Since the liner 100 is being rotated by the mandrel 300, the coating material can be effectively spread circumferentially about the unmasked portion of the liner 100 and onto and circumferentially about the radial skirt portion. As the user requires additional coating material, the user simply deposits additional coating material onto the inner applicator surface and then applies the coating material to the rotating liner 100. The applicator 399 is preferably formed of a flexible material, such as a rubber or other polymeric materials, to permit the applicator 399 to flex as it is placed against the rotating liner 100.

After the coating material is applied to the fabric liner 100 and is pressed into the body thereof, the coating is then preferably further leveled with a use of a roller 531 or the like as shown in FIG. 17. For example, the roller 531 can be a short nap roller that is configured to level the coating.

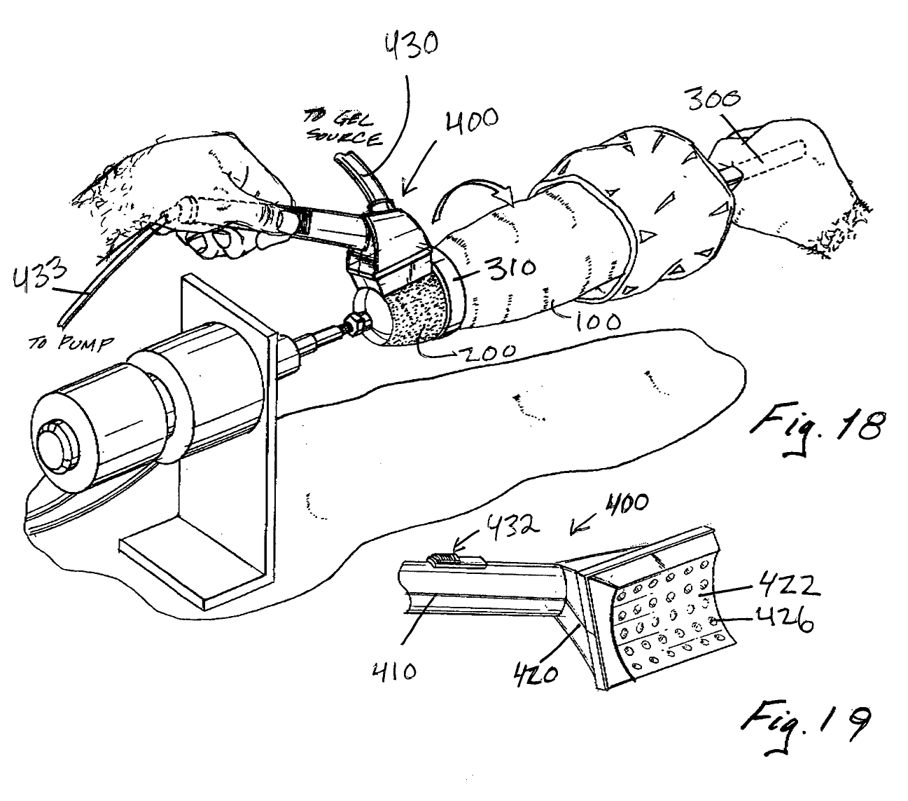

FIGS. 18-19 illustrate one exemplary applicator 400 for applying the coating material 200 on the distal end region of the liner 100 and about the pin receptacle 150. The applicator 400 is a hand held applicator that a user can easily grasp and manipulate as the liner 100 is rotating on the mandrel 300 so as to apply the coating material onto the unmasked portions, while at the same time applying sufficient force (pressure) to ensure that a portion of the coating material is disposed (seeps) into the fabric liner 100.

The applicator 400 has a handle portion 410 to be grasped by the user and an applicator body portion 420 that is coupled to the handle portion 410. The body portion 420 includes an inner applicator surface 422 which receives the coating material and is sufficiently rigid yet flexible to permit the user to effectively press the coating material into the interior of the fabric liner 100. The body portion 420 can and preferably does have some flexibility to permit the body portion 420 to complement the contour of the liner 100 as the user applies the coating material to the curved surfaces of the liner 100.

In other words, the body portion 420 preferably has some degree of flexibility so that it can assume an arcuate shape that is more complementary to the generally annular nature of the liner 100 as compared to a rigid block like body. This permits the body portion 420 to better conform to the annular shape of the liner 100 as the liner 100 is rotated by the mandrel 300 and the coating material is applied. The coating material is applied to the inner applicator surface 422 using any number of different techniques.

For example, the body portion 420 is modified so that it has a fluid delivery mechanism for delivering a discrete amount of coating material to the inner applicator surface 422. For example, the body portion 420 can have a number of openings 426 formed therein and dispersed across the surface 422 for discharging coating material onto the inner applicator surface 422 as best shown in FIG. 19. More specifically, the body portion 420 is in communication with a source of the coating material so that the coating material is fed through a conduit (e.g., tubing) or the like 430 to the body portion 420 where the coating material is distributed to all of the openings 426 through which the material flows onto the inner applicator surface 422. The handle portion 410 can include some type of actuator mechanism 432, such as a trigger or button or the like, that causes the selective discharge of a quantity of the coating material through the openings 426. For example, the body portion 420 can include a valve, etc. that is operatively connected to the actuator mechanism 432 such that when the user manipulates the actuator mechanism 432, the valve is either opened or closed and the coating material advances through the body portion 420 and through the openings 426 when the valve is open until a sufficient amount of coating material is on the inner applicator surface 422 at which time, the user will close the valve with the actuator mechanism 432. The coating material that is on the inner applicator surface 422 is then applied to the liner 100 and the pin receptacle 150 in the manner described above.

The actuator mechanism 432 is preferably electronically based and therefore a wire 433 for delivering control signals to the actuator mechanism 432 extends between the mechanism 432 and a power source (not shown) that is operatively connected to a pump (not shown) or the like for forcibly driving the coating material through the conduit 430. In other words, when the user presses or otherwise actuates the mechanism 432, a control signal is sent to a controller or the like which then instructs the pump to be actuated, thereby causing the coating material to be forced through the conduit 430 and through the openings 426. When the mechanism 432 is deactivated, the pump is instructed to stop applying pressure to the coating material in the conduit 430 and therefore, the coating material is not delivered through the openings 426.

The applicator 100 is systematically operated to continue to discharge the coating material onto the liner 100 and the pin receptacle 150 until a sufficient quantity of coating material is disposed about the liner 100 and the pin receptacle 150. At such time, the coating material is further processed as described below by uniformly spreading the coating material across the exposed surface.

As described herein, some of the preferred coating materials are made upon the mixing of two or more separate chemicals and therefore, the mixing of these chemicals to form the coating material needs to be taken into consideration. For example, one preferred class of coating materials are polyurethanes; as is known, polyurethanes can be formed by mixing isocyanates with a compound or mixture of compounds that containing functional groups that react with isocyanates (e.g., polyfunctional alcohols or polyols; polyfunctional primary or secondary amines, etc.). Thus, the conduit that leads to the body portion 420 can be configured so that the two chemicals are carried separately and then mixed at a location proximate the body portion 420 but far enough away from the body portion 420 so that proper mixing can occur and the desired coating material is formed prior to it being fluidly delivered to the body portion 420. For example, one feed line (conduit) can carry one chemical, while another feed line (conduit) carries the other chemical and the two feed lines join into one single conduit line at a location upstream of the body portion 420 to permit proper mixing and formation of the coating material prior to the coating material being delivered to the body portion 420.

After the coating material is applied to the fabric liner 100 and is pressed into the body thereof, the coating is then further leveled with the roller 531. The roller 531 can either be a manual roller that the user grasps and spreads the coating material with or the roller can be a part of an automated device such that the roller selectively rotates when the device is actuated. When the roller is an automated roller, the liner 100 can be manipulated so that the roller contacts the entire surface of the coating 200 as by rotating the liner 100 itself or by keeping the liner 100 stationary and moving the automated roller around the coating 200 so as to contact the entire surface of the coating 200 with the roller.

The coating material is then cured by applying heat or by allowing the chemical reaction to take place. For example, a number of suitable coating materials, as described below, cure at room temperature by permitting the chemical reaction to take place in this setting.

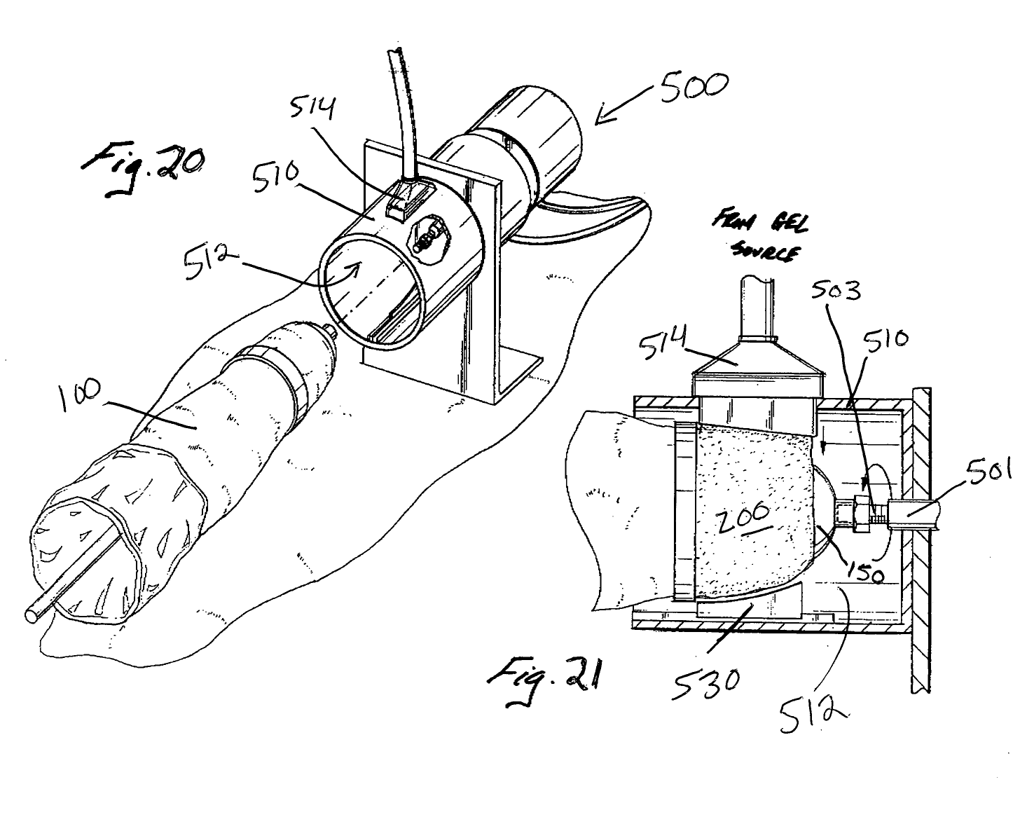

In yet another embodiment, the applicator can be more automated than the aforementioned embodiment. One exemplary embodiment of an automated applicator 500 is illustrated in FIGS. 20-21. In this embodiment, the applicator 500 includes a housing 510 that is constructed so as to receive the distal end of the liner 100 that has been masked off. The housing 510 therefore has an interior cavity 512 that receives the distal end of the liner 100 and permits the liner 100 to be rotated by means of a device 501 that is incorporated into the design of the housing 510. For example, the device 501 is similar or identical to the rotatable lathe in the previous embodiment. The device 501 thus has a rotatable drive axle or shaft 503 that is threaded or otherwise contoured so that a releasable yet secure coupling can take place between the pin receptacle 150 and the shaft 503 so that when the shaft 503 is rotated, the liner 100 is likewise rotated. The shaft 503 protrudes through an opening formed in the housing 510 with an interface between the shaft 503 and the housing 510 containing a gasket or the like so that coating material cannot seep into and through this opening.

When the liner 100 is coupled to the shaft 503, the mandrel 300 is supported so that the liner 100 remains generally horizontal along its length as the distal end is in the housing 510. For example, the mandrel 300 can be hand-held or it can be supported by a mechanical device that retains the mandrel 300 while permitting free rotation thereof or the mandrel can simply rest within a groove formed in a support beam that is of a proper height such that the liner 100 remains generally horizontal.

It will be appreciated that there is a slight gap between the outer surface of the liner 100 and the inner surface of the housing 510 and it is within this gap that the coating material is received and distributed about the liner 100 to form the coating 200.

Within the housing 510 there is one or more injector devices 514 that serve to inject the coating material into the cavity 512 and within the gap between the liner 100 and the inner surface of the housing 510. The injector devices 514 are controllable so that the coating material is not continuously injected into the gap but rather controlled discrete amounts of coating material can be injected into the gap to permit the user to construct a coating having a desired thickness. The precise location of the injector devices 514 is not critical; however, one preferred location for the injector devices 514 is at a location that is between a 10 o'clock and 2 o'clock position relative to the liner 100.

Within the cavity 512 of the housing 510, a wiper or blade assembly 530 is provided for metering the coating material to ensure that the coating material is substantially distributed over the entire exposed area and that the coating 200 has a substantially uniform thickness. The wiper assembly 530 is positioned downstream of where the coating material is injected so that after the coating material is deposited onto the surface of the liner 100 and the liner 100 is rotated, the material contacts the wiper assembly 530 which not only presses the coating material into the interior of the fabric liner 100 to ensure that the coating 200 is properly adhered to the fabric liner 100 but also, the wiper assembly 530 serves to distribute the coating material in a more even manner over the surface of the fabric liner 100. The wiper assembly 530 is preferably adjustable so that the distance between the wiper and the outer surface of the liner 100 is variable and thus, the thickness of the resulting coating 200 is variable to a degree. For example, the wiper assembly 530 can be a spring loaded device that can be adjusted to cause the wiper assembly 530 to move forward and away from the liner 100.

After the coating material has been applied to the liner 100 and the pin receptacle 150 and the wiper assembly 530 has generally spread the coating material over the entire exposed surface, the coating 200 can be subjected to a roller, as described above, for the purpose of further leveling the coating 200.

In this embodiment, the applicator 500 is essentially a completely automated system that is in communication with a master controller or the like that monitors and instructs the various movements of the operating components of the applicator 500. For example, the mandrel on which the liner 100 is placed can be associated with a robotic device which is actuated to position the liner 100 within the cavity 512 and then later withdraws the liner 100 from the cavity 512 after the coating 200 has been formed on the liner 100. In addition, The robotic device can drive the mandrel into a mating connection with the shaft 503. After the liner with coating 200 is removed from the applicator 500, the robotic device can then deliver the liner to another station, such as a curing station, where the coating 200 sits for a predetermined period of time at a predetermined temperature that are sufficient to cause the coating material to cure or otherwise harden and become rigidly adhered to the fabric liner 100.

It will be appreciated that while the above-described applicators are suitable for applying the coating material to the liner 100, there are a number of other techniques that can be used to apply the coating material. For example, the coating material can be applied by a spraying process in which a hand-held sprayer or an automated sprayer is used to apply the coating material to the exposed area. Conventional sprayers that can be used include pressure gun and spatter gun devices as well as air spray equipment with pressure cup gun or a spatter gun. It will be appreciated that with many, if not all, of the sprayers, the coating material mix must be removed and the equipment cleaned before it sets in the equipment, including the conduits (tubing) through which the material is pumped.

In addition, the coating material can simply be brushed onto the fabric liner or it can be rolled on using a roller so long as the coating material is sufficiently pressed into the interior of the fabric to adhere the coating material to the liner 100. It is also possible for the coating material to be applied using molding techniques. For example, the exposed portion of the liner 100 on which coating material is to be applied can be placed into a mold and then the coating material is injected into the mold, thereby forming an injection molded coating 200 of prescribed thickness. Compression molding techniques likewise can be used and in this instance, the coating material is pressed into contact with the liner 100.

As previously mentioned, the liner 100 with coating 200 formed as a part thereof can further limit stresses of a distal end elongation and rotation in a prosthetic pocket. By applying the distal end coating in accordance with the present invention, the following benefits are obtained: (1) the coating creates a composite that can better resist stretching; (2) it allows for a better limb control resulting from reduced axial and rotational movement; (3) it distributes anti-elongation forces in a 360° manner; and (4) it reinforces sewn seams in areas of greatest stress and lessens the risk of fabric tears.

The following example illustrates the application of a coating to a liner according to one embodiment and is meant only to be illustrative and not limiting in any way.

### EXAMPLE

A liner is provided and a mask is placed on the liner such that an area is exposed where the coating is to be formed. The liner is then placed on a mandrel that is operatively coupled to a device (e.g., a lathe) that can selectively rotate the liner. The liner includes the pin receptacle. The rotating device is actuated so that the liner is rotated at a speed between about 30-40 RPM.

The coating material is prepared and is a polyurethane material commercially available under the trade name Poly 74-40 from Polytek of Easton, Pa. This material is a two part RTV liquid rubber (polyurethane mold rubber) that consists of part A and part B, which after mixing, cure overnight at room temperature to flexible high strength RTV mold rubbers. Typically, 2 parts A is mixed with 1 part B to form the coating material. The curing of Poly 74-40 can be accelerated with the addition of a predetermined amount (e.g., up to 3%) of 74/75 Part X (by weight of total mix), which is also available from Polytek. The pour time is about 20 minutes for this particular material; however, it will be appreciated that the pour times can vary considerably depending upon the material being used.

A sufficient amount of Poly 74-40 is made and is then applied to the exposed (unmasked) area of the liner using any type of applicator, such as those disclosed herein. The unmasked area includes the entire radial flange of the pin receptacle and a predetermined area of the distal end of the liner that extends to the pin receptacle. For example, about the bottom 1½ inch of the liner as measured up from the inner circumferential edge of the pin receptacle can be left exposed for receiving the coating material. As the liner rotates, the Poly 74-40 material is dispersed onto the rotating liner and the applicator is used to smooth and spread the material out over the exposed area as well as apply pressure to the coating material to ensure that the Poly 74-40 material adheres to the fabric liner by being pressed into the interior of the fabric as well as the seams of the liner. After the coating material has been generally uniformly dispersed over the unmasked area, a roller is used to further smooth the coating material. After the coating has been satisfactorily applied to the liner, the coating material is then cured. For example, the liner with coating can be stored overnight at room temperature to effectuate the cure. Alternatively, an accelerator can be added to the coating material and the liner is then placed in a heating device, such as an oven, and heated at an elevated temperature (e.g., 150° F.) for a predetermined period of time, e.g., ½ hour.

When the coating is in the form of one or more strips as shown in FIG. 10, the length of each strip is preferably between about 1 inch and 7 inch; however, these lengths are merely exemplary and not limiting. In one embodiment, two distinct strips are disposed along the distal end portion and are spaced about 180° apart from one another. Preferably, the two strips are formed on the two side seams of the fabric liner body, which are oriented about 180° from one another. However, it will be understood that more than two strips can be formed at the distal end portion. For example, four strips can be formed and spaced at predetermined intervals from one another (e.g., 90° apart from one another with two being placed over the side seams).

The size of the cushioned liner 100 can be varied depending upon the dimensions of the residual limb to be enclosed by simply proportionally varying the dimensions of the pattern which is used to cut and form each of the first and second side panels 160, 170 and the distal panel 180. In other words, the length of the cushioned liner 100 of any of the embodiments disclosed herein can vary and the cushioned liner 100 can easily be manufactured in a number of different sizes by simply altering the dimensions of the patterns used to form the first and second fabric side panels 160, 170 and the distal piece 180. In one exemplary embodiment, the cushioned liner 100 has a length between about 8 inches and about 20 inches. Typically, the cushioned liner 100 is constructed to have a prescribed length and can then be modified for the individual wearer by simply cutting and removing an upper portion of the article. In this manner, the cushioned liner 100 can be initially produced to have a length that fits or can be easily modified to fit a large percentage of the potential users.

The fabric liner also preferably has a cushioned material that is formed therein as fully described in the commonly assigned '299 U.S. patent application and in commonly assigned U.S. patent application Ser. No. 10/102,377, filed Mar. 19, 2002, which is hereby incorporated by reference in its entirety. These patent applications also describe in detail several methods that can be used to apply the cushioning material to the liner body.

The present application thus provides liners that advantageously are constructed so that they limit the overall stretch and increase wear characteristics by adding a surface reinforcement to a cushion or pin suspension type prosthetic liner. This is accomplished before, after or as a part of the process to add mechanical attachments to fabrics.

While the invention has been particularly shown and described with reference to preferred embodiments thereof, it will be understood by those skilled in the art that various changes in form and details may be made therein without departing from the spirit and scope of the invention.

1. A liner for covering a residual limb of an amputee comprising:
a sock-shaped fabric member; and
a stretch limiting element incorporated into an exterior surface of the sock-shaped fabric member at a distal end portion thereof and formed of a material that has less elasticity than the sock-shaped fabric member such that the elasticity of the distal end portion is reduced to provide surface reinforcement and for limiting stresses of distal end elongation.

2. A liner for covering a residual limb of an amputee comprising:
a fabric liner body formed of at least two fabric pieces, one of the fabric pieces being a distal end piece that is attached to at least one other fabric piece along a circumferential edge of the distal end piece, the distal end piece being free of a transverse seam extending across the distal end piece; and
a coating formed of a material that is integrally adhered to an exterior surface of a distal end region of an outer surface of the fabric liner body for providing surface reinforcement thereat and for limiting stresses of distal end elongation and rotation in a prosthetic socket.

3. The liner of claim 2, wherein the fabric member is formed of first and second side pieces that are attached to one another along vertical edges thereof, the distal end piece having an annular shape such that the distal end piece is attached to one end of each of the first and second side pieces to produce a circumferential seam.

4. The liner of claim 3, wherein each of the first and second side pieces has an elongated generally rectangular shape.

5. The liner of claim 3, wherein the first and second side pieces are stitched to one another along the vertical edges thereof.

6. The liner of claim 3, wherein the distal end piece is attached to the first and second side pieces by a circumferential stitched seam.

7. The liner of claim 2, wherein the fabric member is formed of at least two polymeric materials.

8. The liner of claim 7, wherein the fabric member is formed of a knit of polyester fibers and polypropylene fibers, the polyester fibers formed a first side of the fabric member and the polypropylene fibers forming a second side of the fabric member.

9. The liner of claim 8, wherein the coating is formed on the first side of the fabric member.

10. The liner of claim 2, wherein the material has a durometer hardness between about 10 to about 70 (Shore A) after the material is cured or sets to form the coating.

11. The liner of claim 2, wherein the coating is formed in a complete circumference around the distal end of the liner so as to distribute anti-elongation forces in a 360° manner.

12. The liner of claim 2, wherein the coating is confined to an area occupying a distalmost ½ inch to 4 inches of the fabric liner.

13. The liner of claim 2, wherein the material is a material selected from group consisting of: polyurethanes; liquid silicones; polyamides; rubber latices; and mixtures thereof.

14. The liner of claim 2, wherein the coating has a thickness from about 0.010 inch to about 0.090 inch.

15. The liner of claim 2, wherein the material comprises a material that can be applied under ambient conditions.

16. The liner of claim 2, wherein the coating is applied over one or more seams formed where the at least two fabric pieces are joined to one another.

17. The liner of claim 2, wherein the fabric liner includes an open end and a closed distal end.

18. The liner of claim 2, further including:
a pin receptacle that is attached to the distal end of the liner body on an exterior thereof.

19. The liner of claim 18, wherein said pin receptacle includes a skirt surround a receptacle body, the coating being applied over the skirt and onto the distal end region of the fabric liner.

20. The liner of claim 19, wherein the coating is disposed 360° around the skirt.

21. The liner of claim 19, wherein the receptacle body is a rigid metal member and includes a threaded bore.

22. The liner of claim 2, wherein the coating has a wave-like pattern.

23. The liner of claim 2, wherein the coating comprises a plurality of discrete elongated strips of the flexible material with one end of the strip being adhered to a pin receptacle that is attached to the distal end of the fabric liner on an exterior thereof and an opposite end terminating in the distal end region of the fabric liner.

24. The liner of claim 23, wherein each strip has a generally rectangular shape.

25. The liner of claim 23, wherein each strip has a length between about 1½ inch and about 6 inch.

26. The liner of claim 2, wherein the coating comprises a plurality of discrete elongated strips of flexible material with one end of the strip terminating proximate or at an inner circumferential edge of a pin receptacle that is attached to the distal end of the fabric liner and an opposite end terminating along the fabric liner body.

27. The liner of claim 2, further including:
a pin receptacle that is attached to the distal end of the liner body on an exterior thereof, the pin receptacle including a skirt surrounding a receptacle body with the coating being applied on the exterior of the skirt and onto the distal end region of the fabric liner anywhere from a distance between about 1½ inch to about 4 inch above an upper peripheral edge of the pin receptacle.

28. A liner for covering a residual limb of an amputee, the liner comprising:
a fabric liner body having formed of at least two fabric pieces, one of the fabric pieces being a distal end piece that is attached to at least one other fabric piece along a circumferential edge of the distal end piece, the distal end piece being free of a transverse seam extending across the distal end piece;
a pin receptacle that is attached to the distal end of the liner body on an exterior thereof; and
a first stretch limiting element that is disposed at least partially on the pin receptacle and extends onto and is adhered to a distal section of the liner body; the stretch limiting element being formed of a coating that has limited flexibility and is integrally adhered to an outer surface of the fabric liner body for providing 360° of surface reinforcement of the fabric liner as well as distributing anti-elongation forces in a 360° manner.

29. The liner of claim 28, further including:
a second stretch limiting element that is disposed at or near a proximal section of the liner body, the stretch limiting element being formed of a coating that has limited flexibility and is integrally adhered to an outer surface of the fabric liner body along at least a partial circumference thereof for providing surface reinforcement of the fabric liner as well as limiting the amount of circumferential stretch of the liner.

30. A liner for covering a residual limb of an amputee comprising:
a fabric liner body formed of at least two fabric pieces, one of the fabric pieces being a distal end piece that is attached to at least one other fabric piece along a circumferential edge of the distal end piece, the distal end piece being free of a transverse seam extending across the distal end piece; and
a coating formed of a material that is integrally adhered to an exterior surface of a proximal end region of an outer surface of the fabric liner body for providing surface reinforcement thereat and for limiting circumferential stretch of the fabric liner body, the coating having limited flexibility and extends along at least a partial circumference of the fabric liner body.

31. The liner of claim 30, further including:
a stretch limiting element that is adhered to a distal section of the liner body; the stretch limiting element being formed of a coating that has limited flexibility and is integrally adhered to an outer surface of the fabric liner body for providing 360° of surface reinforcement of the fabric liner as well as distributing anti-elongation forces in a 360° manner.

32. A method of providing surface reinforcement at a distal end of a liner for covering a residual limb of an amputee and for limiting stresses associated with distal end elongation of the liner, the method comprising the steps of:
providing the liner which comprises a fabric liner body including an open end and a closed distal end; and
applying a material to a distal end region of the fabric liner body to form a coating that has limited flexibility and is integrally adhered to the fabric liner body to form an anti-elongation coating on an exterior surface of the liner, the coating having a hardness between about 10 and about 70 durometer (Shore A).

33. The method of claim 32, further including the step of:
disposing the liner on an outer surface of a rotatable mandrel prior to applying the coating; and
rotating the mandrel as the material is applied to the fabric liner body.

34. The method of claim 33, wherein the mandrel is rotated at a speed between about 20 and about 60 RPM.

35. The method of claim 32, further including the step of:
masking the fabric liner body to define an exposed area where the material is to be applied to form the coating.

36. The method of claim 35, wherein the step of masking the fabric liner body includes the step of placing a mask material 360° around the fabric liner body.

37. The method of claim 35, wherein the step of masking the fabric liner body includes the step of placing a mask material 360° around the fabric liner body so that up to a bottommost 6 inches of the fabric liner body is exposed.

38. The method of claim 32, wherein the step of applying the material comprises the step of:
placing the material 360° around the fabric liner body so that the resulting coating extends 360° around the fabric liner body.

39. The method of claim 32, wherein the step of applying the material comprises the step of:
applying the material with sufficient pressure to cause the material to seep into the interior of the fabric liner body resulting in the coating being integrally adhered to the fabric liner body.

40. The method of claim 39, wherein the step of applying the material comprises the step of:
providing an applicator;
disposing an amount of the material on a selected portion of the fabric liner body;
rotating the fabric liner body; and
applying a force with the applicator against the material to cause the material to seep into the interior of the fabric liner body and into any seams of the fabric liner body.

41. The method of claim 40, wherein the applicator comprises a squeegee.

42. The method of claim 40, wherein the applicator comprises an at least partially automated device including a handle portion and a body portion that includes a plurality of openings formed therein for discharging the material onto an applicator surface of the body portion, the material being delivered to the plurality of openings through a conduit that is at least partially disposed in the handle portion.

43. The method of claim 42, wherein the applicator body portion comprises a flexible member.

44. The method of claim 40, wherein the applicator comprises an automated device that includes a housing for receiving the fabric liner body therein and a rotatable drive shaft at least partially extending within the housing and configured to be operatively coupled to a pin receptacle associated with the liner for controlled rotation thereof, the housing having a mechanism for discharging the material onto the fabric liner body and a wiper for smoothing the material and for applying force to the material to cause the material to become integrally disposed within the fabric liner body.

45. The method of claim 32, further including the steps of:
smoothing the material so that it has a substantially uniform thickness;
exposing the fabric liner body with the material to conditions to cause the formation of the coating; and
curing the material to form the coating.

46. The method of claim 32, wherein the step of providing the fabric liner body includes the step of:
attaching a pin receptacle on the distal end of the fabric liner body, the pin receptacle including at least a partially flexible skirt portion.

47. The method of claim 32, wherein the step of applying the material includes the step of:
applying the material circumferentially about the skirt portion and the distal end region of the fabric liner body.

48. The method of claim 32, wherein the step of providing the fabric liner body includes the steps of:
providing at least two fabric pieces, one of the fabric pieces being a distal end piece; and
attaching the distal end piece to at least one other fabric piece along a circumferential edge of the distal end piece, the distal end piece being free of a transverse seam extending across the distal end piece.

49. The method of claim 32, wherein the fabric liner body is formed of first and second side pieces that are attached to one another along vertical edges thereof, the distal end piece having an annular shape such that the distal end piece is attached to one end of each of the first and second side pieces to produce a circumferential seam.

50. The method of claim 32, wherein the fabric liner body is formed of a knit of polyester fibers and polypropylene fibers, the polyester fibers formed a first side of the fabric liner body and the polypropylene fibers forming a second side of the fabric liner body.

51. An automated method of providing surface reinforcement at a distal end of a liner for covering a residual limb of an amputee and for limiting stresses associated with distal end elongation of the liner, the method comprising the steps of:
providing the liner which comprises a fabric liner body including an open end and a closed distal end;
rotating the liner; and
selectively applying a material to a distal end region of the fabric liner body with an automated applicator to form a coating that has limited flexibility and is integrally adhered to the fabric liner body to form an anti-elongation coating on an exterior surface of the liner, the coating having a hardness between about 10 and about 70 durometer (Shore A), wherein the automated applicator has a flexible applicator body for pressing the material against the fabric liner body and a plurality of ports formed therethrough for selectively discharging the material, the applicator having an on-off switch to permit selective discharge of the material.
